(19) United States
(12) Patent Application Publication
(10) Pub. No.: US 2022/0310225 A1
(43) Pub. Date: Sep. 29, 2022

(54) HEALTH MANAGEMENT APPARATUS AND METHOD FOR PROVIDING HEALTH MANAGEMENT SERVICE BASED ON NEUROMUSCULOSKELETAL MODEL

(71) Applicant: ELECTRONICS AND TELECOMMUNICATIONS RESEARCH INSTITUTE, Daejeon (KR)

(72) Inventors: Hang Kee KIM, Daejeon (KR); Ki Suk LEE, Daejeon (KR); Dongchun LEE, Daejeon (KR)

(73) Assignee: ELECTRONICS AND TELECOMMUNICATIONS RESEARCH INSTITUTE, Daejeon (KR)

(21) Appl. No.: 17/506,991

(22) Filed: Oct. 21, 2021

(30) Foreign Application Priority Data
Mar. 24, 2021 (KR) 10-2021-0037963

(51) Int. Cl. G16H 20/30; G16H 30/40; G16H 50/50; G16H 50/20; G06F 30/23; G06T 19/00; A61B 5/11

(52) U.S. Cl. CPC G16H 20/30; G16H 30/40; G16H 50/50; G16H 50/20; G06F 30/23; G06T 19/00; A61B 5/1126; G16H 40/67

(57) ABSTRACT

A health management apparatus based on a neuromuscular skeletal model receives a medical image of a user, and models a skeleton, an external shape, and a muscle of the user in three dimensions based on the medical image to generate a neuromusculoskeletal model of the user. The health management apparatus receives motion data generated based on a motion of the user, and updates the neuromusculoskeletal model based on the motion data. The health management apparatus performs a simulation on the updated neuromusculoskeletal model, and generates an exercise or rehabilitation program of the user based on a result of the simulation.

### CROSS-REFERENCE TO RELATED APPLICATION

This application claims priority to and the benefit of Korean Patent Application No. 10-2021-0037963 filed in the Korean Intellectual Attribute Office on Mar. 24, 2021, the entire contents of which are incorporated herein by reference.

### BACKGROUND

### Field

The described technology relates to a health management apparatus and method for providing a health management service based on a neuromusculoskeletal model.

### DESCRIPTION OF THE RELATED ART

Services that present exercise and rehabilitation programs through mobile devices, personal computers, televisions, etc., and monitor results of the users performing the programs are provided as services to improve the health of users. The users' health cares are useful when they are continuous and can be performed in daily life. The exercise and rehabilitation programs so far have adopted a service in which the user directly selects the exercise program, instead of a customized service according to the health condition of the user.

The user can perform the rehabilitation program with the help of a therapist in the medical facility when the user's movement is inconvenient due to the disease. However, it is difficult for the user to perform the rehabilitation program depending on the health condition after returning home, resulting in that it is accompanied by the inconvenience of frequent visits of the therapist or frequent visits to the hospital.

### SUMMARY

Some embodiments may provide a health management apparatus based on a neuromusculoskeletal model and a method of providing a health management service for generating a user-customized exercise or rehabilitation program based on a user's three-dimensional (3D) model and a user's motion.

According to an embodiment, a neuromuscular skeletal model-based health management apparatus including a memory configured to one or more instructions and a processor configured to execute the one or more instructions may be provided. By executing the one or more instructions, the processor may receive a medical image of a user, model a skeleton, an external shape, and a muscle of the user in three dimensions based on the medical image to generate a neuromusculoskeletal model of the user, receive motion data generated based on a motion of the user, update the neuromusculoskeletal model based on the motion data, perform a simulation on the updated neuromusculoskeletal model, and generate an exercise or rehabilitation program of the user based on a result of the simulation.

In some embodiments, the processor may assign a physical attribute to the neuromusculoskeletal model based on a skeletal structure or body information of the user, and assign a neuromusculoskeletal attribute to the neuromusculoskeletal model based on a change in the muscle and skeleton of the user depending on information transmitted from nerves.

In some embodiments, the processor may perform a simulation on the updated neuromusculoskeletal model based on the physical attribute and the neuromusculoskeletal attribute to analyze the muscle or a joint of the user, and generate the exercise or rehabilitation program based on an analysis result obtained by analyzing the muscle or the joint.

In some embodiments, the physical attribute may include an attribute for a dynamic physics simulation, and the simulation may include the dynamic physics simulation.

In some embodiments, the processor may perform the dynamic physics simulation to analyze an activation degree of the muscle, and the analysis result may include information related to activation of the muscle.

In some embodiments, the physical attribute further includes an attribute for a finite element method (FEM) simulation, and the simulation may further include the FEM simulation. In this case, the processor may perform the dynamic physics simulation to analyze a movement of the joint and a load amount of the joint, perform the FEM simulation by applying the movement and the load amount to the neuromusculoskeletal model, and predict an occurrence of a joint disease based on a result of the FEM simulation. Further, the analysis result may further include a prediction result for the occurrence of the joint disease.

In some embodiments, the processor may provide data representing the exercise or rehabilitation program to a terminal of the user.

In some embodiments, the processor may receive a performance result of the exercise or rehabilitation program from the terminal of the user.

In some embodiments, the processor may model the skeleton and the external shape of the user in three dimensions based on the medical image to generate a 3D model of the user, and model the muscle of the user based on the 3D model to generate the neuromusculoskeletal model.

In some embodiments, the processor may estimate a cartilage based on the 3D model of the skeleton, and the neuromusculoskeletal model may further include the 3D model of the cartilage.

According to another embodiment, a method of providing a health management service performed by a computing device may be provided. The method may include generating a neuromusculoskeletal model of a user based on a medical image of the user, receiving motion data generated based on a motion of the user, updating the neuromusculoskeletal model based on the motion data, performing a simulation on the updated neuromusculoskeletal model, generating an exercise or rehabilitation program of the user based on a result of the simulation, and providing data representing the exercise or rehabilitation program to the user.

In some embodiments, generating the neuromusculoskeletal model may include assigning a physical attribute to the neuromusculoskeletal model based on a skeletal structure or body information of the user, and assigning a neuromusculoskeletal attribute to the neuromusculoskeletal model based on a change in a muscle and a skeleton of the user.

In some embodiments, generating the neuromusculoskeletal model may further include performing a dynamic physics simulation on the updated neuromusculoskeletal model based on the physical attribute and the neuromusculoskeletal attribute to analyze the muscle or a joint of the user, and generating the exercise or rehabilitation program based on an analysis result obtained by analyzing the muscle or the joint.

In some embodiments, generating the neuromusculoskeletal model may further include analyzing a movement of the joint and a load amount of the joint based on the dynamic physics simulation, and performing a finite element method (FEM) simulation by applying the movement and the load amount to the neuromusculoskeletal model. In this case, the analysis result may further include a performance result of the FEM simulation.

According to yet another embodiment, a neuromuscular skeletal model-based health management apparatus including a memory configured to one or more instructions and a processor configured to execute the one or more instructions may be provided. By executing the one or more instructions, the processor may receive a medical image of a user, model a skeleton and an external shape of the user in three dimensions based on the medical image, model a muscle of the user in three dimensions based on 3D models of the skeleton and the external shape, model a cartilage of the user in three dimensions based on the medical image or the 3D model of the skeleton, assign a neuromusculoskeletal attribute to 3D models of the skeleton, the external shape, the muscle, and the cartilage based on a change in the muscle and skeleton of the user depending on information transmitted from nerves of the user to generate a neuromusculoskeletal model of the user, and generate an exercise or rehabilitation program of the user based on the neuromusculoskeletal model.

In some embodiments, the processor may generate the exercise or rehabilitation program by updating the neuromusculoskeletal model based on motion data generated based on a motion of the user.

According to some embodiments, a user-customized exercise or rehabilitation program may be generated based on the user's 3D model and the user's motion.

### BRIEF DESCRIPTION OF THE DRAWINGS

FIG. 1 is an example block diagram of a neuromusculoskeletal model-based health management system according to an embodiment.

FIG. 2 is an example block diagram of a neuromusculoskeletal generating apparatus according to an embodiment.

FIG. 3 is an example flowchart of a method of providing a health management service according to an embodiment.

FIG. 4 is an example flowchart of a neuromusculoskeletal generating method according to an embodiment.

FIG. 5 is an example flowchart of a method of generating an exercise or rehabilitation program according to an embodiment.

FIG. 6 is a diagram for explaining an example of generating an exercise or rehabilitation program according to an embodiment.

FIG. 7 is a diagram showing an example of a computing device according to an embodiment.

### DETAILED DESCRIPTION OF THE EMBODIMENTS

In the following detailed description, only certain example embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

As used herein, the singular forms “a”, “an” and “the” are intended to include the plural forms as well, unless the context clearly indicates otherwise.

The sequence of operations or steps is not limited to the order presented in the claims or figures unless specifically indicated otherwise. The order of operations or steps may be changed, several operations or steps may be merged, a certain operation or step may be divided, and a specific operation or step may not be performed.

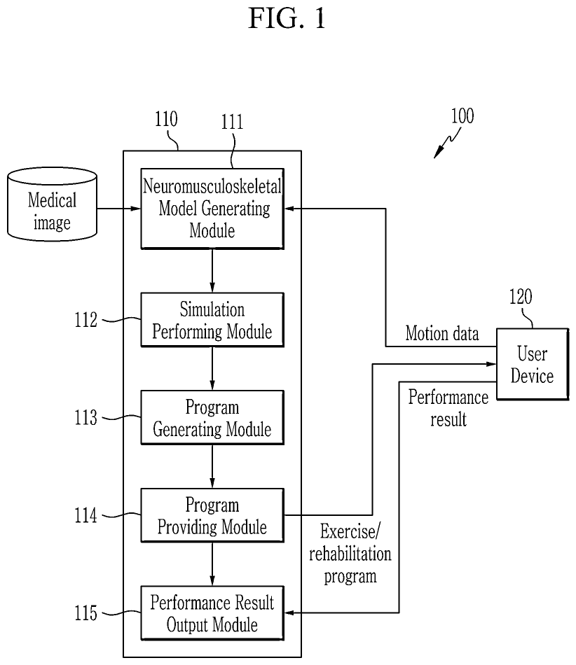

FIG. 1 is an example block diagram of a neuromusculoskeletal model-based health management system according to an embodiment.

Referring to FIG. 1, a neuromusculoskeletal model-based health management system includes a neuromusculoskeletal model-based health management apparatus 110 and a user device 120.

The health management apparatus 110 includes a neuromusculoskeletal model generating module 111, a simulation performing module 112, a program generating module 113, and a program providing module 114.

The neuromusculoskeletal model generating module 111 receives a medical image of a user, and generates a three-dimensional (3D) neuromusculoskeletal model of the user by modeling a body skeleton and external shape of the user in three dimensions based on the medical image. In some embodiments, the neuromusculoskeletal model generation module 111 may assign physical attributes to a 3D model of the user based on a skeleton structure and body information of the user. In some embodiments, the neuromusculoskeletal model generation module 111 may generate neuromusculoskeletal attributes by analyzing changes in muscles and skeletons depending on changes in information transmitted from nerves, and assign the generated neuromusculoskeletal attributes to the 3D model, thereby generating the neuromusculoskeletal model

The simulation performing module 112 performs a simulation based on the 3D neuromusculoskeletal model. In some embodiments, the simulation performing module 112 may perform the simulation based on the 3D neuromusculoskeletal model, the physical attributes, and the neuromusculoskeletal attributes. The program generating module 113 generates an exercise or rehabilitation program of the user based on the simulation performance result, and the program providing module 114 provides data representing the generated program to the user device 120.

In some embodiments, the user device 120 (e.g., a gesture recognition and visualization device) may generate motion data of the user by capturing the user's motions in real-time during daily activities of the user. In this case, the neuromusculoskeletal model generating module 111 may update the 3D neuromusculoskeletal model and the neuromusculoskeletal attributes based on the motion data.

In some embodiments, the user device 120 may reproduce the user's exercise or rehabilitation program to allow the user to perform the exercise or rehabilitation program. The user device 120 may provide the user's performance result on the exercise or rehabilitation program to the health management apparatus 110. In some embodiments, the health management apparatus 110 may further include a performance result output module 115 for outputting the performance result.

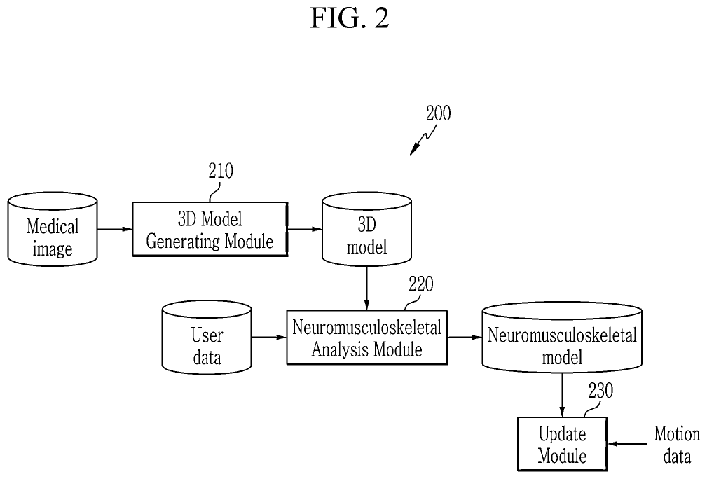

FIG. 2 is an example block diagram of a neuromusculoskeletal generating apparatus according to an embodiment.

In some embodiments, a neuromusculoskeletal generating apparatus 200 shown in FIG. 2 may correspond to a neuromusculoskeletal model generating module 111 shown in FIG. 1.

Referring to FIG. 2, the neuromusculoskeletal generating apparatus 200 includes a 3D model generating module 210, a neuromusculoskeletal analysis module 220, and an update module 230.

The 3D model generating module 210 receives a medical image of a user. In some embodiments, the medical image may include an X-ray image, a computed tomography (CT) image, or a magnetic resonance imaging (MRI) image of the user. The 3D model generating module 210 generates a 3D model by modeling a body skeleton and an external shape in three dimensions based on a medical image. In some embodiments, the 3D model generation module 210 may update the 3D model by modeling muscles based on the 3D model of the external shape and the skeleton. In some embodiments, the 3D model generating module 210 may further generate a cartilage model of the user.

The neuromusculoskeletal analysis module 220 analyzes a physical attribute of each part based on the skeleton structure and body information (e.g., age, weight, etc.) of the user, and provides the physical attributes to the 3D model. The neuromusculoskeletal analysis module 220 generates neuromusculoskeletal attributes by analyzing changes in the muscles and skeletons depending on changes in information transmitted from nerves, and generates a neuromusculoskeletal module by assigning the neuromusculoskeletal attributes to the 3D model.

The update module 230 updates the neuromusculoskeletal model by changing the posture of the neuromusculoskeletal model according to motions indicated by motion data of the user.

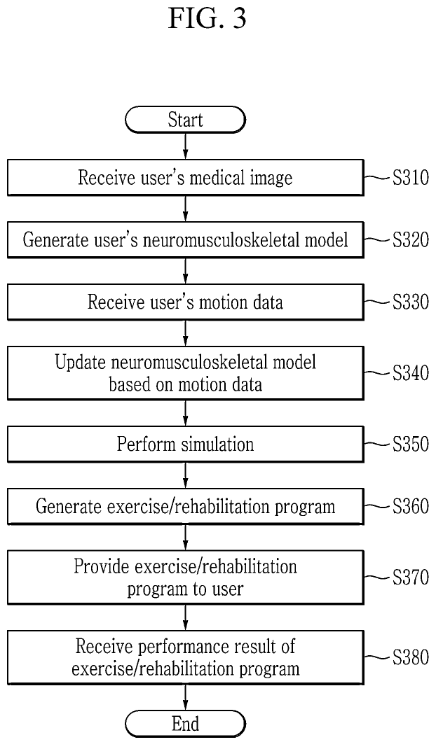

Next, a method of providing a health management service according to various embodiments is described with reference to FIG. 3 to FIG. 6.

FIG. 3 is an example flowchart of a method of providing a health management service according to an embodiment.

Referring to FIG. 3, a computing device receives a medical image of a user at step S310, and models a body skeleton, a shape, and muscles of the user in three dimensions based on the received medical image of the user to generate a neuromusculoskeletal model of the user at step S320.

The computing device receives daily life motion data of the user from a motion recognition and visualization device of the user at step S330. In some embodiments, the motion recognition and visualization device, which is provided at a remote location in which the user lives, may generate the daily life motion data by capturing the user's motions during the daily life in real-time. In some embodiments, the motion recognition and visualization device may use a wearable sensor that is attached to the user's body and obtains motion information of the user by capturing the user's motions. The wearable sensor may include, for example, an inertial measurement unit (IMU). In some embodiments, the motion recognition and visualization device may use an optical sensor that captures an image of the user through a camera and obtains the motion information of the user by analyzing the captured image. The camera may include, for example, an RGB (red, green, blue) camera or an infrared camera. In some embodiments, the gesture recognition and visualization device may use both the wearable sensor and the optical sensor.

The computing device updates the neuromusculoskeletal model by reflecting the motion data in the neuromusculoskeletal model of the user at step S340. In some embodiments, the computing device may update the neuromusculoskeletal model by changing the posture of the neuromusculoskeletal model according to the motion indicated by the motion data.

The computing device performs a simulation based on the updated neuromusculoskeletal model at step S350, and analyzes the user's muscles or joints based on the simulation result, and generates an exercise or rehabilitation program of the user based on the analysis result at step S360.

In some embodiments, the computing device may transmit data representing the generated exercise or rehabilitation program to a user's terminal through a network at step S370. The user's terminal may be, for example, a smart phone, a computer, a television, or a virtual reality or augmented reality device (e.g., a head mounted display (HMD)). In some embodiments, the basic data or execution application of the exercise or rehabilitation program content may be provided and installed in the terminal in advance in order to reduce the network load. In this case, only content setting data for performing the generated exercise or rehabilitation program may be transmitted.

The user may perform the exercise or rehabilitation program received through the terminal, and may derive a performance result obtained by performing the exercise or rehabilitation program through the motion recognition and visualization device. In some embodiments, the user's terminal may provide the performance result of the exercise or rehabilitation program to the computing device through the network at step 5380. Accordingly, the computing device may continuously manage the user's health by tracking the amount of change in exercise or rehabilitation ability.

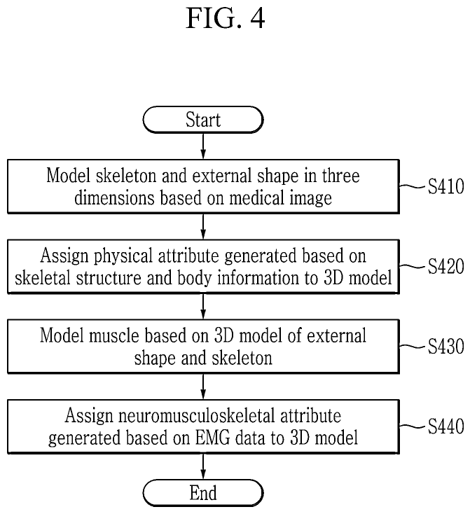

FIG. 4 is an example flowchart of a neuromusculoskeletal generating method according to an embodiment.

In some embodiments, a neuromusculoskeletal generating method shown in FIG. 4 may correspond to step 5320 in a method of providing a health management service shown in FIG. 3.

Referring to FIG. 4, a computing device models a body skeleton and external shape of a user in three dimensions based on the received medical image of the user at step 5410. Since the computing device uses the medical image, the computing device may generate not only an external model but also a skeletal model inside the body as a 3D model. In some embodiments, the computing device may further generate a cartilage model as the 3D model.

In some embodiments, the medical image may include a CT image or an MRI image. Since the CT image or the MRI image is an image obtained by sequentially taking cross-sections of the user's body, the computing device may generate the 3D model by segmentation of a desired part (e.g., spine, pelvis, femur, etc.). In some embodiments, the computing device may automatically perform segmentation of the medical image using deep learning.

In some embodiments, the medical image may include an X-ray image. Since the X-Ray image is not an image obtained by sequentially taking cross-sections of the user's body, the computing device may segment a desired part from the X-Ray image and generate the 3D model that fits a boundary for each part made by the segmentation. In some embodiments, the computing device may perform the segmentation through contour detection. In some embodiments, the computing device may model the 3D model by performing statistical shape model (SSM) optimization to find the 3D model that fits the boundary for each part.

On the other hand, since the cartilage is invisible in the X-Ray image, the 3D model of the skeleton and external shape may be modeled based on the SSM optimization from the X-Ray image, but the cartilage may not be modeled through such the method. Therefore, in some embodiments, the computing device may generate the 3D model of the cartilage by estimating the cartilage based on the 3D model of the skeleton. For example, when the 3D models of the femur and tibia are generated, the computing device can determine the joint skeletal contour of the knee region composed of the femur and shin bone, and the skeletal shape of the knee joint region and the Cartilage can be estimated through the skeletal gap. In some embodiments, the computing device may automatically estimate the cartilage from a skeletal shape and skeletal spacing of a joint part using deep learning.

Next, the computing device analyzes associated physical attributes based on the user's data, and assigns the physical attributes to the 3D model at step S420. In some embodiments, the user's data may include a skeletal structure and body information (e.g., age, weight, etc.) of the user. In some embodiments, the computing device may assign the physical attribute corresponding to each part of the 3D model. In some embodiments, the physical attributes may include attributes for a dynamic physics simulation or attributes for a finite element method (FEM) simulation. The attributes for dynamic physics simulation may include information of each part, for example, volume, weight, or inertia tensor. The attributes for FEM simulation may include, for example, strain, Young's modulus, or viscosity.

Further, the computing device updates the 3D model by modeling muscles based on the 3D model of the external shape and skeleton at step S430. For example, the computing device may model the quadriceps muscle connected to the femur as four muscles that are located in front of the femur and are connected to the pelvis, the femur, and the patella. The computing device generates neuromusculoskeletal attributes by analyzing changes in the muscles and skeleton depending on changes in information transmitted from nerves, and assigns the neuromusculoskeletal attributes to the 3D model at step S440. In some embodiments, the computing device may analyze changes in the muscles and skeleton depending on the changes in information transmitted from nerves based on the user's EMG data generated by an electromyography (EMG) sensor.

In this way, the computing device may generate a 3D neuromusculoskeletal model of the user by integrating the neuromusculoskeletal attributes into the 3D model of the skeleton, the external shape, and the muscles.

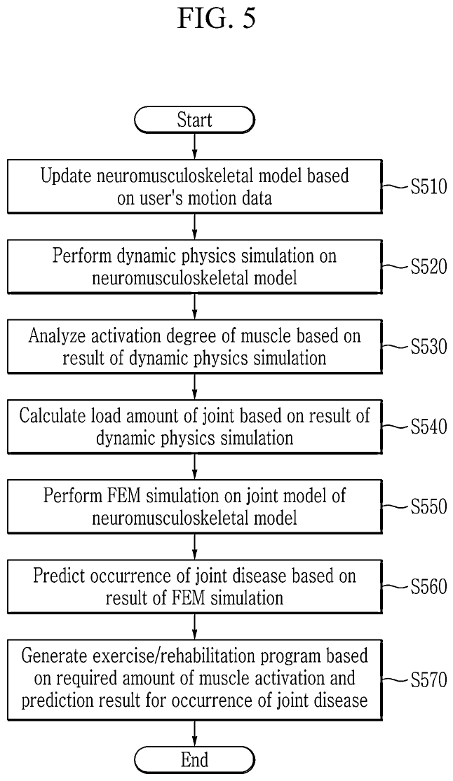

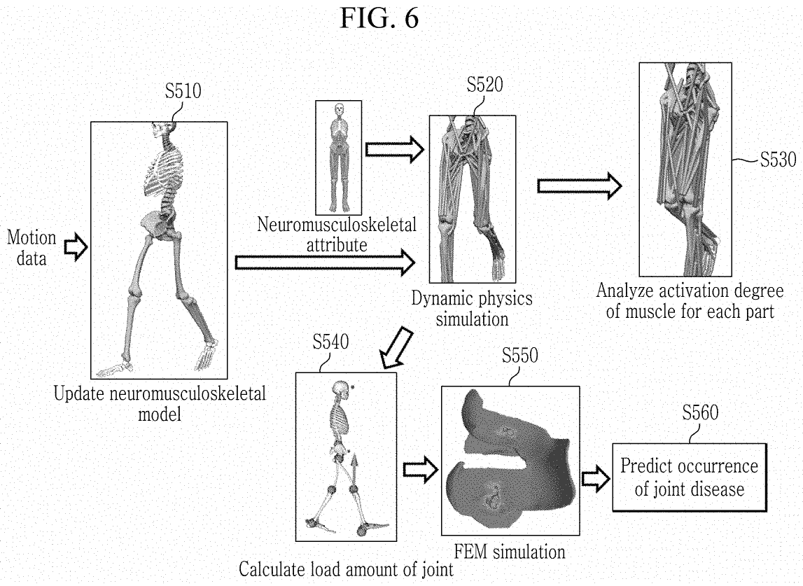

FIG. 5 is an example flowchart of a method of generating an exercise or rehabilitation program according to an embodiment, and FIG. 6 is a diagram for explaining an example of generating an exercise or rehabilitation program according to an embodiment.

In some embodiments, a method of generating an exercise or rehabilitation program shown in FIG. 5 may correspond to steps S340 to S360 in a method of providing a health management service shown in FIG. 3.

Referring to FIG. 5 and FIG. 6, a computing device updates a neuromusculoskeletal model based on user's motion data at step S510. In some embodiments, since the user's motion data indicates a user's motion, for example, a position and angle of each joint, the computing device may change the posture of the neuromusculoskeletal model over time according to the user's motion data.

The computing device performs a simulation on the neuromusculoskeletal model whose posture is updated based on physical attributes and neuromusculoskeletal attributes at step S520. In some embodiments, the simulation performed in step S520 may include a dynamic physics simulation, and the dynamic physics simulation may be performed based on attributes for dynamic physics simulation among the physical attributes. In some embodiments, the neuromusculoskeletal attributes may include a length, angle, force or activation degree of each muscle, an amount of load imparted to each joint, or a movement of each joint (e.g., an amount of change in a position or angle). Therefore, as a result of performing the dynamic physics simulation, some of the neuromusculoskeletal attributes may be updated. The computing device may analyze the activation degree of each muscle from among the updated attributes to determine information related to a required amount of muscle activation at step S530. In some embodiments, the computing device may analyze whether the muscles are abnormally activated due to a posture abnormality of the user by analyzing the activation degree of each muscle, and whether there is a muscle requiring activation based on the abnormally inactivated muscles. Accordingly, the computing device may determine information related to the required amount of muscle activation.

Further, the computing device may calculate the load amount of each joint based on a result of the dynamic physics simulation at step S540, and apply the load amount of each joint to the joint model of the neuromusculoskeletal model to perform the simulation at step S550. In some embodiments, the simulation performed in step S550 may include an FEM simulation of each joint, and the FEM simulation may be performed based on attributes for FEM simulation among the physical attributes. In some embodiments, the computing device may further apply the motion of each joint to the joint model of the neuromusculoskeletal model in addition to the load amount of each joint. The computing device calculates a load amount or tensile strength for each detailed element of each joint based on a result of the FEM simulation to predict an occurrence of a disease in each joint at step S560. In some embodiments, the computing device may predict the current or future occurrence of the joint disease. The joint disease may be, for example, osteoarthritis.

The computing device generates an exercise or rehabilitation program of the user based on the required amount of muscle activation and the prediction result for the occurrence of the joint disease at step S570. For example, when the user is using only one arm due to hemiplegia or arthritis, the computing device may generate the exercise or rehabilitation program capable of assisting a movement of an opposite arm with no movement in order to assist the rehabilitation of the opposite arm. In some embodiments, the exercise or rehabilitation program may include a method of directly guiding a specific movement to be performed by the user or a method of reproducing specific content (e.g., game, entertainment, etc.) to allow the user to voluntarily exercise the specific movement. For example, in a case of using the game content that drives a car by manipulating a steering wheel, when there is an arm part that needs a rehabilitation exercise due to hemiplegia or arthritis, the computing device may identify a movement of the steering wheel that can induce a movement of the arm part and generate a driving course based on the identified movement. In some embodiments, the exercise or rehabilitation program may be manually generated by a program manager, or may be automatically generated by using optimization simulation or deep learning, based on the required amount of muscle activation and the prediction result for the occurrence of the joint disease.

According to the above-described embodiments, a user-customized exercise or rehabilitation program may be generated and provided to the user based on the user's neuromusculoskeletal model and motion data. Accordingly, the user can continuously perform health management, and also perform the health management in daily life. Further, it is possible to increase the efficiency of health promotion by continuously monitoring the user's current state and providing the exercise or rehabilitation program suitable for the current state. Furthermore, it is possible to eliminate the inconvenience of frequent visits of a therapist or frequent visits to a hospital for the user who needs rehabilitation due to the disease, by performing the health management remotely.

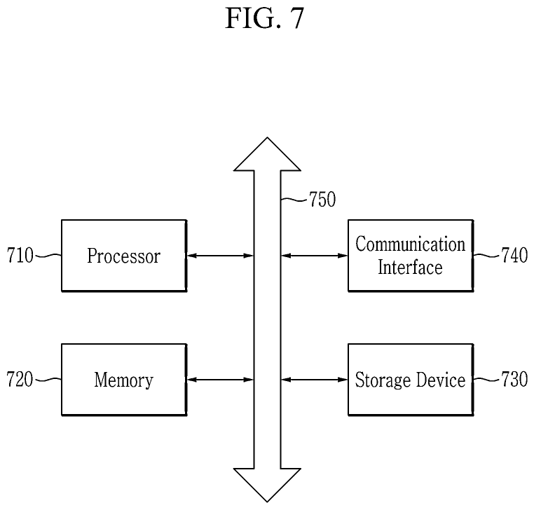

Next, an example computing device for implementing a neuromusculoskeletal model-based health management apparatus or a method of providing a health management service according to embodiments is described with reference to FIG. 7.

FIG. 7 is a diagram showing an example of a computing device according to an embodiment.

Referring to FIG. 7, a computing device includes a processor 710, a memory 720, a storage device 730, a communication interface 740, and a bus 750. The computing device may further include other general components.

The processor 710 controls the overall operation of each component of the computing device. The processor 710 may be implemented with at least one of various processing units such as a central processing unit (CPU), a microprocessor unit (MPU), a micro controller unit (MCU), and a graphic processing unit (GPU), or may be implemented with a parallel processing unit. Further, the processor 710 may perform operations on a program for executing a method of providing a health management service or functions of a neuromusculoskeletal model-based health management apparatus described above.

The memory 720 stores various data, instructions, and/or information. The memory 720 may load a computer program from the storage device 730 to execute the method of providing the health management service or the functions of the neuromusculoskeletal model-based health management apparatus. The storage device 730 may non-temporarily store the program. The storage device 730 may be implemented as a non-volatile memory.

The communication interface 740 supports wireless communication of the computing device.

The bus 750 provides a communication function between components of the computing device. The bus 750 may be implemented as various types of buses such as an address bus, a data bus, and a control bus.

The computer program may include instructions that cause the processor 710 to perform the method of providing the health management service or the functions of the neuromusculoskeletal model-based health management apparatus when loaded into the memory 720. That is, the processor 1110 may perform the method of providing the health management service or the functions of the neuromusculoskeletal model-based health management apparatus by executing the instructions.

The method of providing the health management service or the functions of the neuromusculoskeletal model-based health management apparatus may be implemented as a computer-readable program on a computer-readable medium. In some embodiments, the computer-readable medium may include a removable recording medium or a fixed recording medium. In some embodiments, the computer-readable program recorded on the computer-readable medium may be transmitted to another computing device via a network such as the Internet and installed in another computing device, so that the computer program can be executed by another computing device.

While this invention has been described in connection with what is presently considered to be practical embodiments, it is to be understood that the invention is not limited to the disclosed embodiments, but, on the contrary, is intended to cover various modifications and equivalent arrangements included within the spirit and scope of the appended claims.

What is claimed is:

1. A health management apparatus based on a neuromuscular skeletal model, comprising:
a memory configured to one or more instructions; and
a processor configured to, by executing the one or more instructions:
receive a medical image of a user;
model a skeleton, an external shape, and a muscle of the user in three dimensions based on the medical image to generate a neuromusculoskeletal model of the user;
receive motion data generated based on a motion of the user;
update the neuromusculoskeletal model based on the motion data;
perform a simulation on the updated neuromusculoskeletal model; and
generate an exercise or rehabilitation program of the user based on a result of the simulation.

2. The health management apparatus of claim 1, wherein the processor is configured to:
assign a physical attribute to the neuromusculoskeletal model based on a skeletal structure or body information of the user; and
assign a neuromusculoskeletal attribute to the neuromusculoskeletal model based on a change in the muscle and skeleton of the user depending on information transmitted from nerves.

3. The health management apparatus of claim 2, wherein the processor is configured to:
perform a simulation on the updated neuromusculoskeletal model based on the physical attribute and the neuromusculoskeletal attribute to analyze the muscle or a joint of the user; and
generate the exercise or rehabilitation program based on an analysis result obtained by analyzing the muscle or the joint.

4. The health management apparatus of claim 3, wherein the physical attribute includes an attribute for a dynamic physics simulation, and
wherein the simulation includes the dynamic physics simulation.

5. The health management apparatus of claim 4, wherein the processor is configured to perform the dynamic physics simulation to analyze an activation degree of the muscle, and
wherein the analysis result includes information related to activation of the muscle.

6. The health management apparatus of claim 3, wherein the physical attribute further includes an attribute for a finite element method (FEM) simulation,
wherein the simulation may further include the FEM simulation,
wherein the processor is configured to:
perform the dynamic physics simulation to analyze a movement of the joint and a load amount of the joint;
perform the FEM simulation by applying the movement and the load amount to the neuromusculoskeletal model; and
predict an occurrence of a joint disease based on a result of the FEM simulation, and
wherein the analysis result further includes a prediction result for the occurrence of the joint disease.

7. The health management apparatus of claim 1, wherein the processor is configured to provide data representing the exercise or rehabilitation program to a terminal of the user.

8. The health management apparatus of claim 7, wherein the processor is configured to receive a performance result of the exercise or rehabilitation program from the terminal of the user.

9. The health management apparatus of claim 1, wherein the processor is configured to:
model the skeleton and the external shape of the user in three dimensions based on the medical image to generate a three-dimensional (3D) model of the user; and
model the muscle of the user based on the 3D model to generate the neuromusculoskeletal model.

10. The health management apparatus of claim 9, wherein the processor is configured to estimate a cartilage based on the 3D model of the skeleton, and wherein the neuromusculoskeletal model further includes the 3D model of the cartilage.

11. A method of providing a health management service performed by a computing device, the method comprising:
generating a neuromusculoskeletal model of a user based on a medical image of the user;
receiving motion data generated based on a motion of the user;
updating the neuromusculoskeletal model based on the motion data;
performing a simulation on the updated neuromusculoskeletal model;
generating an exercise or rehabilitation program of the user based on a result of the simulation; and
providing data representing the exercise or rehabilitation program to the user.

12. The method of claim 11, wherein generating the neuromusculoskeletal model comprises:
assigning a physical attribute to the neuromusculoskeletal model based on a skeletal structure or body information of the user; and
assigning a neuromusculoskeletal attribute to the neuromusculoskeletal model based on a change in a muscle and a skeleton of the user.

13. The method of claim 12, wherein generating the neuromusculoskeletal model further comprises:
performing a dynamic physics simulation on the updated neuromusculoskeletal model based on the physical attribute and the neuromusculoskeletal attribute to analyze the muscle or a joint of the user; and
generating the exercise or rehabilitation program based on an analysis result obtained by analyzing the muscle or the joint.

14. The method of claim 13, wherein generating the neuromusculoskeletal model further comprises:
analyzing a movement of the joint and a load amount of the joint based on the dynamic physics simulation; and
performing a finite element method (FEM) simulation by applying the movement and the load amount to the neuromusculoskeletal model, and
wherein the analysis result further includes a performance result of the FEM simulation.

15. A health management apparatus based on a neuromuscular skeletal model, comprising:
a memory configured to one or more instructions; and
a processor configured to, by executing the one or more instructions:
receive a medical image of a user;
model a skeleton and an external shape of the user in three dimensions based on the medical image;
model a muscle of the user in three dimensions based on 3D models of the skeleton and the external shape,
model a cartilage of the user in three dimensions based on the medical image or the 3D model of the skeleton,
assign a neuromusculoskeletal attribute to 3D models of the skeleton, the external shape, the muscle, and the cartilage based on a change in the muscle and skeleton of the user depending on information transmitted from nerves of the user to generate a neuromusculoskeletal model of the user; and
generate an exercise or rehabilitation program of the user based on the neuromusculoskeletal model.

16. The health management apparatus of claim 15, wherein the processor is configured to generate the exercise or rehabilitation program by updating the neuromusculoskeletal model based on motion data generated based on a motion of the user.
